(19) United States
(12) Patent Application Publication
(10) Pub. No.: US 2011/0166651 A1
(43) Pub. Date: Jul. 7, 2011

(54) Lens Having Independent Non-Interfering Partial Zones

(76) Inventor: Werner Fiala, Wien (AT)

(21) Appl. No.: 13/062,553

(22) PCT Filed: Aug. 28, 2009
(86) PCT No.: PCT/AT09/00338

§ 371 Date: Mar. 7, 2011

(30) Foreign Application Priority Data
Sep. 9, 2008 (AT) A 1402/2008

(51) Int. Cl. A61F 2/16; G02C 7/02; G02C 7/06; G02C 7/04

(52) U.S. Cl. 623/6.11; 351/159; 351/168; 351/161

(57) ABSTRACT

A lens, particularly a contact lens or intraocular lens, for improving the image quality of incident polychromatic light exhibiting wave front errors, including a central zone and at least one annular zone, is characterized in that such that positive or negative optical wavelength differences exist between adjoining zones of the lens in the direction of the lens axis, said differences being at least as large as the coherence length of polychromatic light.

### FIELD OF THE INVENTION

The invention relates to a lens for improving the imaging quality of incident polychromatic light exhibiting wavefront aberrations, wherein the lens is subdivided into a first central partial zone and at least one second annular partial zone concentric with respect thereto, and wherein between mutually adjacent zones of the lens in the direction of the lens axis there are positive or negative optical path length differences which are at least as large as the coherence length of polychromatic light. The invention relates, in particular, to an ophthalmic lens, preferably an intraocular lens (IOL).

### KNOWN PRIOR ART

Lenses comprising annular zones which contain optical steps between the zones are known, see e.g. EP 470 811 B1. The document U.S. Pat. No. 5,982,543 (Fiala) describes a zone lens in which the area of the individual zones is a maximum of 0.0056*λ, where X is the average wavelength of the light used. The maximum area of the individual zones is therefore 3.08 mm² for λ=550 nm, or 3.92 mm² for λ=700 nm. U.S. Pat. No. 7,287,852 (Fiala) describes a zone lens in which the depth of focus of the individual zones is at least 1.1 diopters.

Furthermore, lenses for the correction or compensation of the wavefront aberrations of an incoming light beam are known. By way of example, WO 01/89424 A1 (Norrby et al.) describes a lens whose refractive surfaces are configured such that it converts a light beam having large wavefront aberrations into a light beam having smaller aberrations. WO 2004/108017 A1 (Fiala et al.) describes a lens which converts a wavefront curved in elliptically oblong fashion, that is to say a wavefront having a wavefront aberration, into a substantially spherical wavefront, that is to say a wavefront having a vanishing wavefront aberration

### BACKGROUND OF THE INVENTION

Ophthalmic lens serve, in interaction with other optical system of the eye, such as cornea and, if appropriate, natural lens of the eye, to image an object point in a conjugate image point, wherein the image point ideally lies on the retina of the eye. It is known that the cornea generally has spherical aberration, i.e. the cornea refracts light rays near the axis more weakly than those far from the axis.

FIG. 1A schematically shows a pseudophakic eye, which substantially consists of the cornea 4 and the IOL 5. IOLs having spherical refracting surfaces 6 and 7 have been used for decades. These spherical lenses themselves have spherical aberration. The optical path lengths 8, 9 and 10 between an object point 1 and the conjugate image point 2 differ in length in the case of a combination of a cornea having spherical aberration and an IOL having spherical aberration. That means that the imaging of such an optical system, that is to say of the pseudophakic eye, is not ideal.

IOLs having aspherical surfaces instead of spherically refracting surfaces have been developed in recent years. Such aspherical IOLs can be embodied in such a way that they have a negative spherical aberration that exactly compensates for the positive spherical aberration of the cornea. Lenses of this type are called “aberration-correcting”. The imaging of the object point 1 into the image point 2 is then diffraction-limited since all path lengths 8, 9 and 10 are identical in magnitude.

Since mass production of IOLs which exactly compensates for the spherical aberration of a specific cornea is not possible - the spherical aberration of the cornea is different for each individual eye—IOLs which compensate for the spherical aberration of an average eye have been developed as a compromise. Such lenses are called “aberration-corrected”. If such an IOL is implanted into an eye whose cornea has a different spherical aberration than the average cornea, then the optical path lengths 8, 9 and 10 are once again different, and the imaging is not diffraction-limited.

Furthermore, IOLs which themselves have no spherical aberration have recently been developed; such lenses, too, have aspherical refracting surfaces 6 and 7. If such “aberration-free” lenses are implanted into an eye whose cornea has spherical aberration, then the optical path lengths 8, 9 and 10 are different in this case, too, which once again means that the imaging is not diffraction-limited.

The above explanations relate to pseudophakic eyes in which the IOLs are situated in an exactly centered and untilted position.

FIG. 1B schematically illustrates a pseudophakic eye with a decentered IOL. As the decentering of the IOL increases, generally the differences in the optical path lengths 8, 9 and 10 increase, and the imaging is generally poorer than with a centered IOL. This statement applies both to spherical, aberration-corrected, aberration-free and to aberration-correcting IOLs.

IOLs in a pseudophakic eye can also be tilted. As can be deduced from the previous explanations, in the case of tilting, too, the optical path lengths 8, 9 and 10 in all of the various lens models are not identical in magnitude. Accordingly, the imaging quality with a tilted IOL is likewise non-ideal.

Since decentering and tilting of an IOL cannot be ruled out in practice, the imaging quality of the optical system, that is to say of the pseudophakic eye, is in practice non-ideal, i.e. not diffraction-limited. The imaging quality of the pseudophakic eye is all the poorer, the greater the differences in the optical path lengths, 8, 9 and 10.

### SUBJECT MATTER OF THE INVENTION

The aim of the invention is an ophthalmic lens which leads to better imaging quality than lenses of a conventional type if the imaging is not diffraction-limited, for example if the lens is decentered or tilted.

The aim of the invention is achieved with a lens, in particular a contact lens or an intraocular lens, comprising a central zone and at least one annular zone, wherein between individual zones of the lens in the direction of the lens axis there are positive or negative optical path length differences which are at least as large as the coherence length of polychromatic light, which lens is characterized in that the surface of each zone is at least 4 mm² in each case.

The lens according to the invention has the advantage of subdividing the wavefront aberration associated with a large diameter of the incident light beam into at least two smaller and mutually independent wavefront aberrations, and of increasing the imaging quality associated with the mutually independent wavefront aberrations by comparison with the imaging quality that can be obtained with the non-subdivided wavefront aberration. The imaging aberrations that occur upon tilting or decentering of the lens are thereby minimized.

Lenses for the correction of wavefront aberrations according to the substantive invention therefore have, within the lens surface, at least one discontinuity of the optical path lengths between an object point and the associated image point. Such discontinuity is achieved either by means of a topographic step on at least one of the lens surfaces or by choosing different optical materials in different partial zones of the lens according to the invention.

According to U.S. Pat. No. 5,982,543, the value of 1 micron (=1 μm) or greater shall be mentioned as typical values for the coherence length of polychromatic light. Said value corresponds to the coherence length of white light.

Lenses according to the substantive invention also have, in particular, zone areas which are significantly greater than the zone areas according to U.S. Pat. No. 5,982,543, i.e. in each case at least 4 mm², and the depth of focus of which is significantly smaller than the value mentioned in U.S. Pat. No. 7,287,852, i.e. preferably in each case at most 1.1 diopters.

Preferably, the lens is subdivided into at least two annular zones of substantially identical refractive power, which do not interfere among one another.

The topographic step mentioned can be achieved, for example, by the central circular lens zone of a conventional lens being recessed slightly in the direction of the lens axis, as a result of which a step arises between the adjoining lens zones and the central thickness of said central zone is less than the central thickness of a conventional lens. If more than one annular lens zone is provided, then topographic steps are likewise provided between the further annular lens zones.

If, alternatively, different optical materials are used in the different partial zones of the lens in order to produce the different optical path lengths, the different optical material can preferably be inserted into a central recessed region or a central cutout of the remaining lens material in order to form said central zone layer there.

Further features and advantages of the invention will become apparent from the appended claims and the following description of preferred embodiments of the invention with reference to the accompanying drawings, which show as follows:

### BRIEF DESCRIPTION OF THE DRAWINGS

FIG. 1A schematically illustrates the essential optical components of a pseudophakic eye. The intraocular lens is centered ideally in this example.

FIG. 1B schematically illustrates once again the essential optical components of a pseudophakic eye. The intraocular lens is now decentered.

FIG. 2 illustrates the resultant amplitudes of a diffraction-limited ideal lens and a non-ideal lens.

FIG. 3A illustrates the resultant amplitude of a conventional lens having spherical refractive surfaces having a diameter of 4.5 mm at the nominal focal point of the lens. Furthermore, FIG. 3A illustrates the partial amplitudes if the lens is subdivided into two partial zones in accordance with the invention.

FIG. 3 illustrates the resultant amplitude of a lens having spherically refractive surfaces having a diameter of 4.5 mm at the nominal focal point of the lens. Furthermore, the partial amplitudes are illustrated if the lens is subdivided into three partial zones in accordance with the invention.

FIG. 4 illustrates an embodiment of an intraocular lens (IOL) according to the invention in cross section.

FIG. 5 illustrates a further embodiment of a contact lens or phakic intraocular lens or intracorneal lens according to the invention, in cross section. Only the optical part of such a lens is shown in FIG. 5.

FIG. 6 illustrates yet another embodiment of a lens according to the invention, in cross section.

FIG. 7 shows the Strehl numbers of a pseudophakic eye with a decentered conventional spherical IOL and with a decentered spherical IOL according to the invention. The figure also contains Strehl numbers which apply to the individual zones of the IOL according to the invention.

FIG. 8 shows the Strehl numbers of a pseudophakic eye with a decentered optimized aspherical IOL and with a decentered aspherical IOL according to the invention. The figure also contains Strehl numbers that apply to the individual zones of the IOL according to the invention.

FIG. 9 shows the Strehl numbers of a pseudophakic eye with a tilted conventional spherical IOL and with a tilted spherical IOL according to the invention. The figure also contains Strehl numbers that apply to the individual zones of the IOL according to the invention.

FIG. 10 shows the Strehl numbers of a pseudophakic eye with a decentered conventional aspherical IOL and with a decentered aspherical IOL according to the invention. The figure also contains Strehl numbers that apply to the individual zones of the IOL according to the invention.

FIG. 11 shows the Strehl numbers of a pseudophakic eye with a decentered conventional aberration-free IOL and with a decentered aberration-free IOL according to the invention.

FIG. 12 shows the Strehl numbers of a pseudophakic eye with a tilted conventional aberration-free IOL and with a tilted aberration-free IOL according to the invention.

### DESCRIPTION OF THE PREFERRED EMBODIMENTS OF THE INVENTION

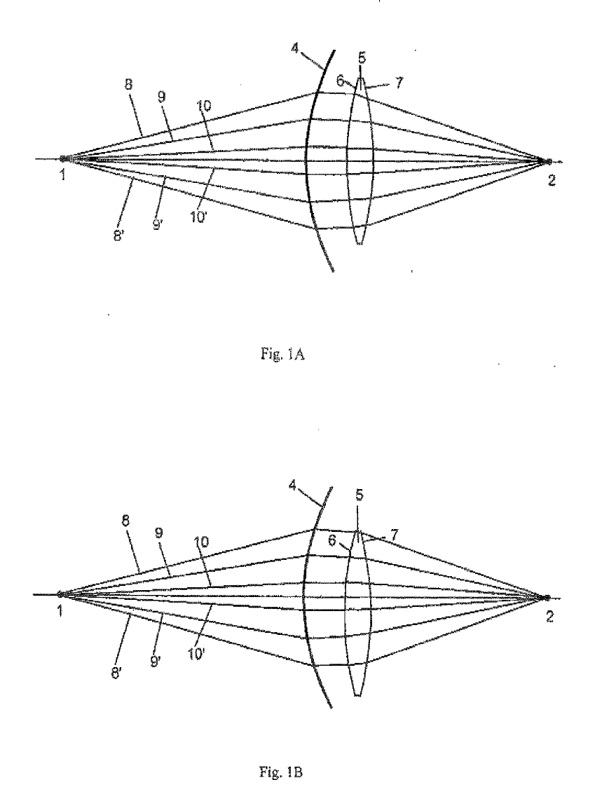

With regard to FIGS. 1A and 1B, reference is made to the explanation in the introductory part of the description.

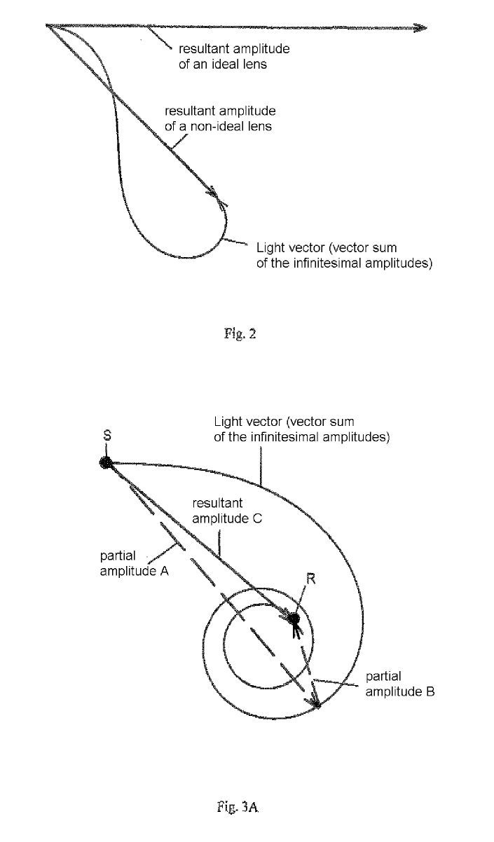

FIG. 2 shows the resultant light vector of an ideal diffraction-limited lens and of a non-ideal lens. In the case of an ideal lens, all the light rays between the object point and the conjugate image point have identical optical path lengths. If such a lens is subdivided into a large number of annular zones, then the infinitesimal amplitudes of the individual zones have the same phase angle or the same direction. The vector sum of all the infinitesimal amplitudes then attains the possible maximum since all the infinitesimal amplitudes have the same direction. In contrast thereto, the optical path lengths between the object point and the conjugate image point in the individual annular zones of a non-ideal lens are different. Therefore, the phase angles of the individual infinitesimal amplitudes are also different and the vector sum of the infinitesimal amplitudes, which is called “light vector” in FIG. 2, is less than that of an ideal lens. A discussion of the phase angles and amplitudes in the case of non-ideal lenses or in defocus positions of lenses can be found in the publication: W. Fiala and J. Pingitzer, “Analytical approach to diffractive multifocal lenses”, Eur. Phys. J. AP 9, 227-234 (2000).

The basic conditions explained with reference to FIG. 2 constitute the starting point for realizing the lens according to the invention.

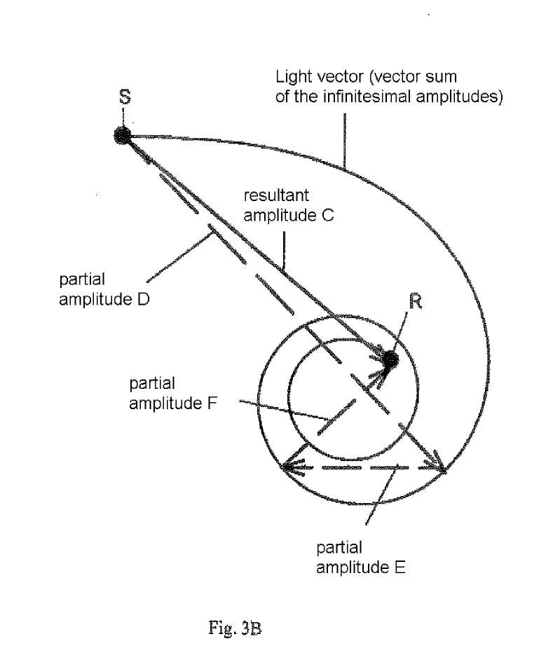

FIG. 3A shows the light vector of a spherical lens at the nominal, i.e. paraxial, focus. As can be seen, the infinitesimal amplitudes have continuously larger phase angles with increasing distance from the lens center S, for which reason the light vector curls up spirally. The resultant amplitude C between S and the lens edge R is therefore small.

If, according to the invention the lens is then subdivided into an inner circular zone and an adjacent annular zone and interference between these two partial zones is suppressed, the resultant partial amplitude of the inner partial zone assumes the value A, and the resultant partial amplitude of the adjacent annular zone assumes the value B. In a known manner, the interference of polychromatic light between the partial zones is suppressed by optical steps that are larger than the coherence length of polychromatic light being introduced between the partial zones. As is known, the value for the coherence length that is applicable to white light is 1 micron. Moreover, reference is made to the corresponding explanations concerning the coherence length of polychromatic light in U.S. Pat. No. 5,982,543.

The light intensity associated with a given amplitude is given by the square of the amplitude. As can be seen, then, from FIG. 3A, the following holds true:

A²+B²>C² (1)

That means that, by subdividing the lens into two independent partial zones, it is possible to increase crucially, if appropriate, the light intensity at the nominal focus.

If the lens according to FIG. 3B is subdivided into three independent partial zones, the following holds true:

D²+E²+F²>C² (2)

This shows that the total intensity of the spherical lens at the nominal focus can also be increased crucially, if appropriate, by subdivision into three independent partial zones.

It is evident that, by subdividing the lens into more than three zones, it is likewise possible to increase the total intensity at the nominal focus of a lens according to the invention. The restrictions with regard to minimum lens zone area and maximum depth of focus of the lens zones in accordance with the abovementioned patent specifications U.S. Pat. No. 5,982,543 and U.S. Pat. No. 7,287,852 should be taken into consideration in the embodiment of lenses according to the invention.

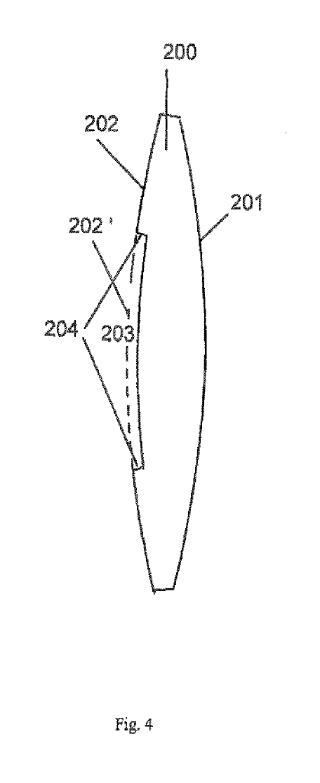

FIG. 4 shows an intraocular lens (IOL) as an example of a lens according to the invention. The IOL 200 has the back surface 201 of a conventional lens. The front surface consists of the annular part 202 and central part 203. Situated between the parts 202 and 203 is a step 204, which is dimensioned such that between the annular lens, consisting of the front surface 202 and the back surface 201, and the central circular lens, consisting of the front surface 203 and the back surface 201, an optical path length difference arises which is larger than the coherence length of polychromatic light. Given a topographic height of the step 204 of t mm, said optical path length difference tₒₚₜ=t* (n_{L}−n₁) mm, where n_{L} is the refractive index of the lens 200 and nᵢ is the refractive index of the immersion medium of the lens. In the case of an IOL, nᵢ is usually given by the value 1.336. If the index n_{L}=1.46, for example, then a step height t of approximately 40 microns is required for an optical path length difference tₒₚₜ of e.g. 5 microns. The refractive powers of the annular lens and of the central circular lens are substantially identical in magnitude.

Owing to the small topographic step height of a few hundredth of a millimeter, the surface 203 can theoretically be formed by the original surface 202′ of a conventional lens being recessed by the absolute value t. The resulting slight reduction of the central thickness of the central lens zone causes only a minimal change in the original refractive power that would be given with the surfaces 202′ and 201.

By means of the procedure just described, it is also possible to convert conventional lenses having toric surfaces into lenses according to the invention having toric surfaces.

Furthermore, the surface 201 can be embodied in such a way that the lens is a bi- or multifocal lens, that is to say that the surface 201 can also have diffractive or refractive bi- or multifocal structures.

FIG. 4 illustrates the step 204 on the front surface of the lens. It goes without saying that said step can also be present on the back surface of a lens in order to prevent interference of polychromatic light between the individual zones of the lens.

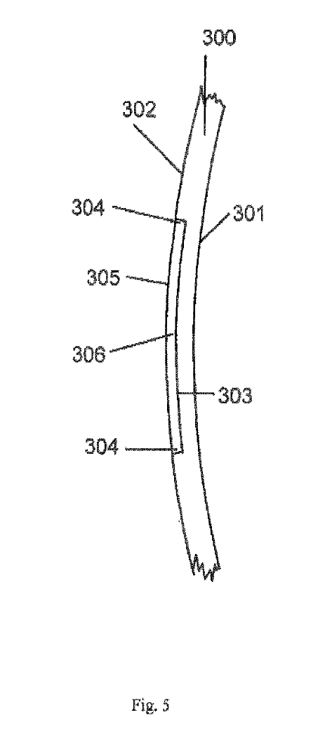

FIG. 5 illustrates in cross section an embodiment of a contact, intracorneal or phakic intraocular lens 300 according to the invention. FIG. 5 shows only the optical part of such a lens. The lens has the back surface 301 of a conventional lens; the front surface of the lens consists of an annular part 302 and a central circular part 303, wherein the circular part 303 is recessed relative to the annular surface 202 by a step 304. An insert lens 306 is placed onto the front surface 303, the back surface of which insert lens is complementary to the surface 303 and the front surface 305 of which insert lens is shaped such that it adjoins the surface 302 continuously at the location of the step 304. The lenses 300 and 306 each have different refractive indices. This gives rise, between the central circular lens and the adjacent annular lens, to an optical path length difference tₒₚₜ=t* (n_{L}−n_{z}), where n_{L} is the index of the lens 300 and n_{z} is the index of the insert lens 306. The step height t should be chosen such that the absolute value of tₒₚₜ is greater than the coherence length of polychromatic light. In a known manner, the curvatures of the surfaces 301, 302, 303 and 305 should be chosen such that the refractive powers in the annular lens and the circular central lens substantially correspond. In the case of a phakic IOL, the insert lens 305 can be omitted, since the immersion medium of the phakic IOL has a different, usually lower, refractive index than the phakic IOL 300.

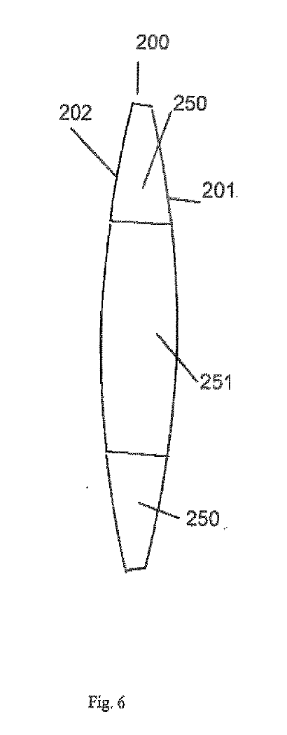

A further possibility for introducing optical path length differences between adjacent lens zones is shown in FIG. 6. A lens 200 is composed of a central circular lens 251 and an annular lens 250. The lens 250 has an index having a different value than the index of the lens 251. From the above statements, it is directly comprehensible to the person skilled in the art that, with such an arrangement between the annular lens 250 and the central lens 251, it is possible to introduce an optical path length difference which is larger than the coherence length of polychromatic light. In order to achieve such a path length difference, the refractive indices of the lens zones 250 and 251 have to differ by a certain absolute value, which is generally very small and amounts to only a few hundredths. The refractive surfaces of the central lens zone 251 and of the annular lens zone 250 should be shaped such that these partial zones have substantially the same refractive power.

### Determining the Imaging Quality of Lenses According to the Invention

As emerges from the discussion of lenses according to the invention with reference to FIGS. 3A and 3B and the above inequalities 1 and 2, it is possible to increase drastically, if appropriate, the total intensity at the focus of such lenses. Better imaging can be achieved as a result of an increased focal intensity.

The Strehl number is frequently used for identifying the imaging quality of lenses or lens systems. As is known, said Strehl number is the ratio of the maximum intensity in the point spread function of the image of a point by a non-ideal lens and the maximum intensity in the point spread function of the image of the same point by an ideal lens. The English expression “point spread function” (PSF) is often used in the German literature as well.

According to the above statements, the Strehl number of an ideal lens or of an ideal lens system is equal to 1. An explanation is given below as to how the Strehl numbers of lenses according to the invention can be calculated.

The intensity of a lens zone i having an area proportion pᵢ of the total area of the lens shall be designated Iᵢ and the Strehl number of said lens zone shall be designated Sᵢ. The Strehl number Sᵢ is equal to the normalized intensity I_{i,n} of the non-ideal lens zone if the intensity of the ideal lens zone of the same size is normalized to 1. Since the intensity is the square of the corresponding amplitude, the following equation holds true for the absolute value of the normalized amplitude A_{i,n} of the lens zone:

|A_{i,n}|=\sqrt{I_{i,n}} (3)

The absolute value of the amplitude of a lens zone is directly proportional to the area proportion of this zone. Therefore, the absolute value of the amplitude of the lens zone is given by:

|Aᵢ|=|A_{i,n}|×pᵢ (4)

and the intensity of the lens zone results as:

Iᵢ=|Aᵢ|²=A_{i,n}²×pᵢ²=I_{i,n}×pᵢ²=Sᵢ×pᵢ² (5)

The normalized total intensity I_{tot,zon} of the lens subdivided into zones is then given by:

$${I}_{tot , zon} = {\sum }_{i} {I}_{i , n} \times {p}_{i}^{2} = {S}_{tot , zon} \qquad ( 6 )$$

The normalized total intensity I_{tot,zon} is equal to the Strehl number of the lens subdivided into i non-interfering independent partial zones.

This intensity or this Strehl number should then be compared with that normalized intensity I_{tot,0} which is achieved with the same lens without zone subdivision, that is to say with a comparable conventional lens. For this purpose, said conventional lens is also subdivided into zones, in which case however, no optical path length differences were introduced between said zones, that is to say that the zones of said lens interfere.

Let us now consider the case where a lens is subdivided into two zones. In the case where the zones interfere, the vectorial total amplitude A_{tot,0} of the lens is given by the vector sum of the individual vectorial amplitudes A₁ and A₂ of the partial zones:

\vec{A}_{tot,0}=\vec{A}₁+\vec{A₂} (7)

and the intensity of said lens having interfering zones is given by:

I_{tot,0}=(\vec{A}₁+\vec{A}₂)²=(\vec{A}_{1,n}p₁+\vec{A}_{2,n}p₂)² (8)

Using equation 3 above, the following is obtained:

I_{tot,0}=I_{1,n}p₁²+I_{2,n}p₂²+2\sqrt{I_{1,n}}\sqrt{I_{2,n}}p₁p₂ cos (φ_{1,2})=I_{tot,zon}+2\sqrt{I_{1,n}I_{2,n}}p₁p₂ cos (φ_{1,2}) (9)

In equation 9, φ_{1,2} is the angle between the vectorial partial amplitudes A₁ and A₂.

The following relationships therefore hold true:

I_{tot,zon}=I_{tot,0} for φ_{1,2}=90° (10a)

I_{tot,zon}>I_{tot,0} for 90°<φ_{1,2}<180° (10b)

I_{tot,zon}>I_{tot,0} for 0°<φ_{1,2}<90° (10c)

The normalized total intensity of the lens subdivided into independent zones is therefore greater than the normalized total intensity of the conventional lens not subdivided into independent zones if the angle between the partial amplitudes is greater than 90 degrees.

The following relationship generally holds true for a lens subdivided into m independent partial zones according to the invention:

$$\begin{matrix}{I}_{tot , 0} = {( {\sum }_{i = 1}^{m} {\vec{A}}_{i , n} {p}_{i} )}^{2} = {I}_{tot , zon} + Remainders \\ or & ( 11 ) \\ {I}_{tot , zon} = {I}_{tot , 0} - Remainders & ( {11}^{′} )\end{matrix}$$

In equations 8 to 11 above, the normalized intensities are identical to the corresponding Strehl numbers.

If the sum of the remainders in equation 11 is positive, then the normalized intensity or the Strehl number of the lens subdivided into the independent zones is less than the Strehl number of the comparable conventional lens having the same diameter. If the sum of the remainders is negative, then the lens according to the invention is superior to the conventional lens.

Evaluations of various lens systems (also see below), in which conventional lenses are compared with lenses according to the invention have revealed that a subdivision according to the invention of a lens into at least two independent partial zones is expedient when the Strehl numbers of a lens system which are attainable with conventional lenses are approximately 0.5 or less.

With the aid of the above considerations, by way of example, the Strehl numbers were determined for some cases of lens systems which alternatively contain conventional IOLs and IOLs according to the substantive invention. By way of example, pseudophakic eyes were investigated as lens systems.

FIG. 7 shows the various Strehl numbers for a pseudophakic eye consisting of a cornea having a central refractive power of 43 diopters and a topographic corneal asphericity of −0.26. Said pseudophakic eye contains a spherical IOL having a refractive power of 20 diopters, which is optionally subdivided into zones according to the invention. In FIG. 7, the Strehl numbers are represented for different lens decentering at the best focus in each case. The Strehl numbers for the two-zone lens according to the invention were determined from the Strehl numbers of the individual lens zones using equation 6 above. The Strehl numbers of the individual lens zones are likewise illustrated in FIG. 7.

In this example, the inner first partial zone has a diameter of 3.5 mm; the second partial zone is a ring lens having an internal diameter of 3.5 mm, which extends as far as the lens edge. The diameter of the light incident on the lens is 5 mm, for which reason, for the substantive calculations, a value of 5 mm is assumed for the external lens diameter. The essential lens parameters are presented in FIG. 7. As can be seen, the spherical IOL subdivided into independent partial zones according to the invention is superior to the conventional spherical IOL in the entire decentering range investigated.

It is stated that the area of the inner partial zone having a diameter of 3.5 mm has the value of 9.6 mm². the second annular partial zone extends as far as the lens edge and has an area of 18.6 mm² given a lens diameter of 6 mm, for example. Given a computational lens diameter of 5 mm, the annular partial zone has an area of 10 mm².

The inner partial zone having a constant refractive power has a depth of focus of 0.36 diopter, and the annular partial zone having an internal diameter of 3.5 mm and an external diameter of 6 mm has a depth of focus of 0.19 diopter. If the value of 5 mm is assumed for the external diameter, then the annular partial zone has a depth of focus of 0.35 diopter.

Recently, IOLs have become known which compensate for the spherical aberration of the cornea, such that (in the absence of other aberrations) the pseudophakic eye is theoretically a diffraction-limited optical system. Such IOLs are called “aberration-correcting”. The Strehl number of a pseudophakic eye equipped with such an IOL is then 1, but only if this IOL is perfectly centered. In the case of decentering of such IOLs, the Strehl number of the pseudophakic eye generally falls significantly. Conventional aberration-correcting IOLs are not the subject matter of the invention, but aberration-correcting IOLs subdivided into independent zones are the subject matter of this invention.

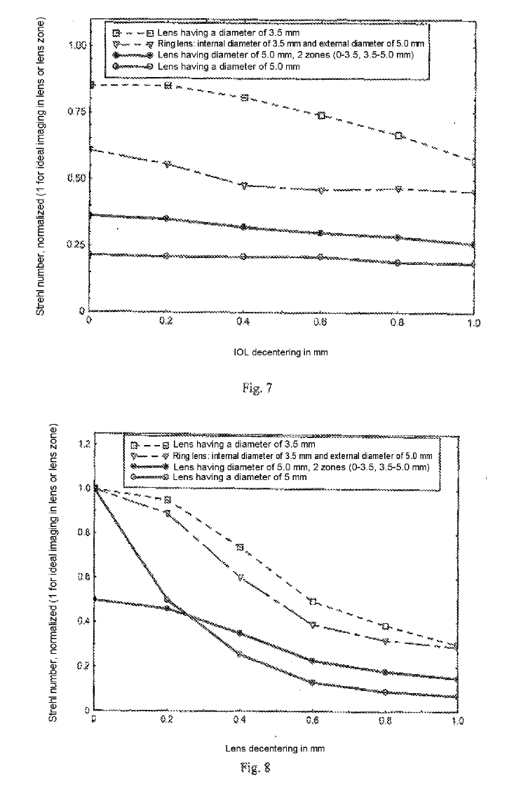

FIG. 8 shows the various Strehl numbers for a pseudophakic eye with an aberration-correcting conventional IOL and an aberration-correcting IOL according to the invention. The IOL according to the invention is subdivided into a central zone lens of 3.5 mm and into a further annular zone lens with 3.5 mm internal diameter, which extends as far as the external diameter of the lens. The diameter of the light incident on the IOL is 5 mm as above. For characterizing the individual zone diameters, the statements made in connection with FIG. 7 are analogously applicable.

As can be seen in FIG. 8, the Strehl number both with the conventional aberration-correcting IOL and with the independent partial zones is equal to 1. Since, with perfect centering, the angle between the two partial amplitudes is zero, the Strehl number of the aberration-correcting IOL according to the invention, given a perfect lens position, is lower than in the case of the conventional IOL. As can be seen, however, the Strehl number of the conventional IOL falls rapidly with increasing decentering, and, above a decentering of approximately 0.25 mm, the IOL according to the invention is superior to the conventional IOL.

The Strehl numbers of pseudophakic eyes also fall if the implanted IOL is tilted with respect to the optical axis of the eye. FIG. 9 shows the Strehl numbers of different lenses in the case of tilting of the IOL. A cornea having a central refractive power of 43 diopters and a topographic asphericity of −0.26 in combination with optionally a conventional spherical IOL having a refractive power of 20 diopters and a spherical IOL according to the invention having the same refractive power is shown as an example. The IOL according to the invention is subdivided into a central zone having a diameter of 3 mm and an annular lens having an internal diameter of 3 mm, wherein the annular lens zone again extends as far as the edge of the IOL. The value 4.5 mm is assumed as the diameter of the light beam incident on the lenses. For the specification of the individual diameters of the lens zones, the statements made in connection with FIG. 7 are applicable. As can be seen from the results in FIG. 9, the IOL according to the invention is superior to the conventional IOL in the entire range of the tilting investigated.

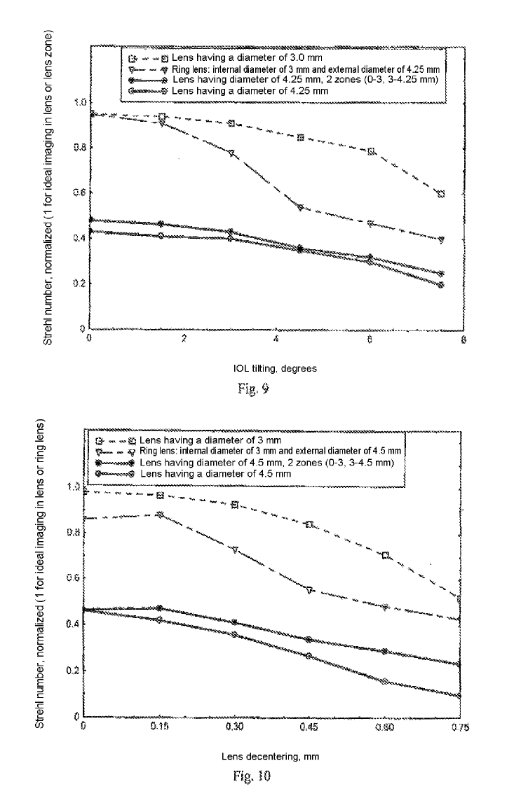

IOLs which compensate for the spherical aberration of an average cornea have been commercially available for some years. Such lenses are called “aberration-corrected”. Examples of such lenses are presented in WO 01/89424 A1 (Norrby et al.) and WO 2004/108017 A1 (Fiala et al.). If such an IOL is implanted into an eye whose spherical aberration does not correspond to that of said average cornea, then the resultant pseudophakic eye is not a diffraction-limited optical system. FIG. 10 shows the Strehl numbers of a pseudophakic eye with increasing decentering of the IOL, in which the cornea has a central refractive power of 47 diopters and a topographic asphericity of -0.03; this cornea is combined with an IOL optimized for an average cornea of 43 diopters and a topographic asphericity of −0.26. This IOL optimized for an average cornea undercompensates for the spherical aberration of the aforementioned cornea of 47 diopters.

The IOL according to the invention consists of a central lens zone having a diameter of 3 mm and an annular lens zone having an internal diameter of 3 mm; said annular lens zone extends as far as the lens edge. The value 4.5 mm is assumed for the diameter of the light beam impinging on the IOL. For the lens diameters specified in FIG. 10, the statements made in FIG. 7 are analogously applicable. It can be discerned from the results in FIG. 10 that the aberration-corrected IOL according to the invention is superior to the conventional aberration-corrected IOL in the entire range investigated for the lens decentering.

It is stated that the inner zone of the lens according to the invention having a diameter of 3 mm, as discussed in FIG. 10, has an area of 7.07 mm² and a depth of focus of 0.49 diopter. The annular partial zone of the lens according to the invention has a minimum area of 8.84 mm² and a maximum depth of focus of 0.39 diopter.

Furthermore, intraocular lenses and also contact lenses are known which have no spherical aberration; these lenses are called “aberration-free”. One example of such lenses can be gathered from US 2005/0203619 A1 (Altmann). Such IOLs are offered for example by the manufacturers Carl Zeiss Meditec, Germany, and Bausch & Lomb, USA. IOLs of this type do not alter the spherical aberration and the other aberrations of the cornea.

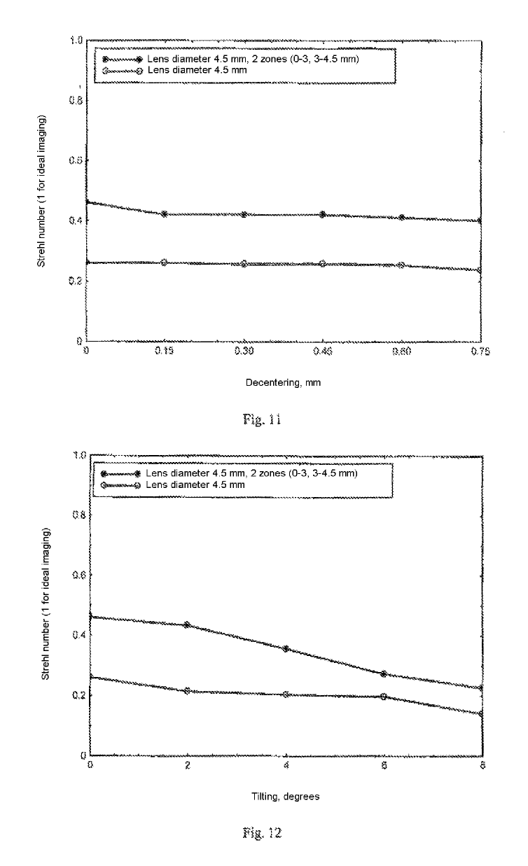

FIG. 11 shows the Strehl numbers of a pseudophakic eye for different lens decenterings of a conventional aberration-free IOL and of an aberration-free IOL according to the invention. The pseudophakic eye in this representation has a cornea having a central refractive power of 47 diopters, and the topographic asphericity of the cornea is −0.03. As can be seen from the values represented in FIG. 11, the aberration-free IOL according to the invention is superior to the conventional aberration-free IOL.

FIG. 12 shows the Strehl numbers of a pseudophakic eye for different lens tilting of a conventional aberration-free IOL and of an aberration-free IOL according to the invention. The pseudophakic eye in this representation has, as before, a cornea having a central refractive power of 47 diopters, and the topographic asphericity of the cornea is once again −0.03. As can be seen from the values represented in FIG. 12, the aberration-free IOL according to the invention is superior to the conventional aberration-free IOL in the case of lens tilting as well.

On the basis of the examples shown it can be seen that lenses according to the substantive invention are generally superior to conventional lenses. One exception to this result is aberration-correcting lenses in a perfect, i.e. ideally centered and untilted, position. The relevant literature reveals that the average decentering of IOLs is approximately 0.2 to 0.25 mm and the average tilting is approximately 2 to 3 degrees. Consequently, the case of an aberration-correcting IOL in a perfect position is rather unlikely.

This shows that generally lenses according to the invention having independent partial zones enable better imaging quality than conventional lenses. This statement applies to spherical and aberration-corrected lenses in a centered and decentered or tilted position to aberration-free lenses, and also to aberration-correcting lenses, provided that the latter have a decentering of a few tenths of a millimeter.

It has furthermore been shown that the partial zones of the lenses described by way of example have areas that are significantly larger than the maximum areas of the zones of zone lenses in accordance with U.S. Pat. No. 5,982,543. Furthermore, the comparatively large partial zones of the lenses according to the invention have a depth of focus which is significantly less than the minimum depth of focus of the zones of a lens in accordance with U.S. Pat. No. 7,287,852.

Lenses according to the invention having two independent partial zones have been described comprehensively by way of example. It is evident to the person skilled in the art that lenses according to the invention can also have a plurality of independent partial zones, provided that the abovementioned restrictions with regard to zone area and depth of focus of the individual zones are fulfilled.

Furthermore, the conditions for intraocular lenses have been discussed comprehensively by way of example. The general insights from this discussion can be applied by the person skilled in the art to the conditions in the case of contact lenses, phakic intraocular lenses and, if appropriate, also intracorneal lenses, that is to say generally to ophthalmic lenses.

What is claimed is:

1. A lens for improving the imaging quality of incident polychromatic light exhibiting wavefront aberrations, comprising a central zone and at least one annular zone, wherein between mutually adjacent zones of the lens in the direction of the lens axis there are positive or negative optical path length differences which are at least as large as the coherence length of polychromatic light, and further wherein the surface area of each zone is at least 4 mm².

2. The lens as claimed in claim 1, wherein the depth of focus of each zone is at most 1.1 diopters.

3. The lens as claimed in claim 1 the individual zones wherein the central zone and the at least one annular zone have substantially the same refractive power.

4. The lens as claimed in claim 1, wherein the difference in the optical path length is produced by a topographic step on at least one surface of the lens.

5. The lens as claimed in claim 4, wherein the central zone is recessed in stepped fashion relative to the annular zone surrounding it.

6. The lens as claimed in claim 4 further comprising more than two zones, wherein an annular zone is recessed in stepped fashion relative to the nearest annular zone surrounding it.

7. The lens as claimed in claim 1, wherein the difference in the optical path length is produced by different optical materials in the different zones.

8. The lens as claimed in claim 7, wherein said different optical material is inserted into a central recessed region of the remaining lens material in order to form said central zone in said region.

9. The lens as claimed in claim 7, wherein said different optical material fills a central cutout of the remaining lens material in order to form said central zone in said region.

10. A lens for improving the imaging quality of incident polychromatic light exhibiting wavefront aberrations, wherein the lens is subdivided into a first central partial zone and at least one second annular partial zone concentric with respect thereto, and further wherein
the surface area of each partial zones is at least 4 mm²;
the depth of focus of each partial zones is less than 1.1 diopters; and
the difference in the optical path lengths through in each case two adjacent partial zones is at least 1 μm in order to prevent interference of polychromatic light passing through the partial zones.

11. The lens as claimed in claim 10, wherein the optical path length difference between the partial zones is produced by a topographic step on at least one of the lens surfaces.

12. The lens as claimed in claim 10, wherein the optical path length difference between the partial zones is produced by different optical materials having different refractive indices that are situated in the partial zones.

13. The lens as claimed in claim 11, wherein the refractive powers in the partial zones of the lens are substantially identical in magnitude.

14. The lens as claimed in claim 1, wherein the lens is a toric lens.

15. The lens as claimed in claim 1, wherein the lens is a bi- or multifocal lens.

16. The lens as claimed in claim 1, wherein the lens is an ophthalmic lens.

17. The lens as claimed in claim 16, wherein the ophthalmic lens is a contact lens.

18. The lens as claimed in claim 16, wherein the ophthalmic lens is one of an intraocular lens, phakic intraocular lens and an intracorneal lens.

19. The lens as claimed in claim 1, wherein the lens is an aberration-correcting, aberration-corrected or aberration-free lens.
